(19) United States
(12) Patent Application Publication
(10) Pub. No.: US 2007/0253569 A1
(43) Pub. Date: Nov. 1, 2007

(54) Communicating with active noise reducing headset

(76) Inventor: Amar G. Bose, Wayland, MA (US)

Correspondence Address: FISH & RICHARDSON PC, P.O. BOX 1022, MINNEAPOLIS, MN, 55440-1022, US

(21) Appl. No.: 11/411,483

(22) Filed: Apr. 26, 2006

(51) Int. Cl. A61F 11/06

(52) U.S. Cl. 381/71.6

(57) ABSTRACT

An active noise reducing headphone has a noise-reducing microphone in a noise-reducing circuit, an outside microphone, a signal input terminal and an outside microphone output terminal and a switch for selectively coupling the headphone to one of the signal input and microphone output terminals.

The present invention relates in general to communicating with a noise reducing headset and more particularly concerns novel apparatus and techniques using an active noise reducing headset having headphones selectively connectable between a connector for receiving external electrical signals and a microphone for transducing speech outside the headset.

### BACKGROUND OF THE INVENTION

For background reference is made to Publication US 2005/0213774 A1 published Sep. 29, 2005, the information set forth in paragraph [002] thereof, pending U.S. application Ser. No. 10/868,318 filed Jun. 15, 2004, now Publication US 2005/0276421A1 published Dec. 15, 2005, and Japanese Unexamined Patent Application Publication No. H8-70493 published Mar. 12, 1996, all incorporated by reference herein.

### SUMMARY OF THE INVENTION

According to the invention, an active noise reducing headset has headphones selectively interconnected between a connector for receiving external electrical signals and a microphone for transducing speech outside the headphones.

Other features, objects and advantages of the invention will become apparent from the following detailed description when read in connection with the accompanying drawing in which:

### BRIEF DESCRIPTION OF THE SEVERAL VIEWS OF THE DRAWING

FIG. 1 is a block diagram of the logical arrangement of a system according to the invention;

FIG. 2 shows a headset with microphones on each headphone receiving outside speech; and

FIG. 3 is a block pictorial diagram of an embodiment of the intention using feed forward active noise reduction.

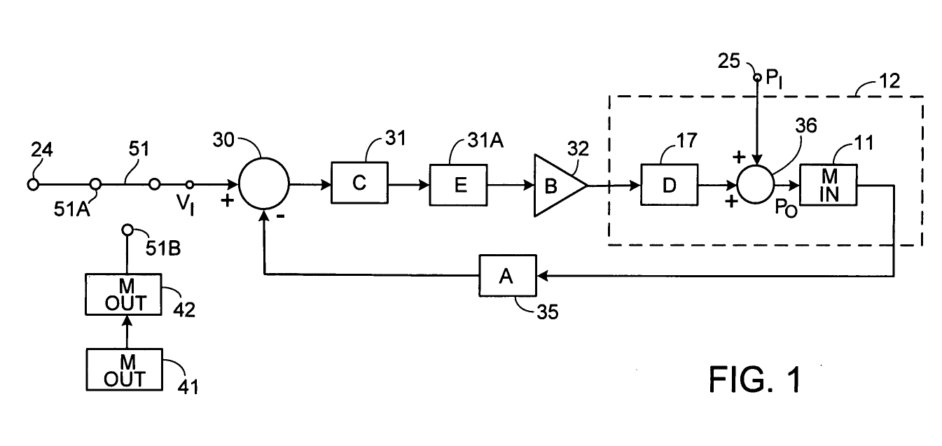

With reference to the drawing and more particularly FIG. 1 thereof, there is shown a block diagram illustrating the logical arrangement of a system according to the invention. In this embodiment a signal combiner 30 algebraically combines the signal desired to be reproduced by the headphone on input terminal 24 connected to switch terminal S1A with switch S1 connected as shown with the feedback signal provided by internal microphone preamplifier 35. Signal combiner 30 provides the combined signal to compressor 31 which limits the level of high level signals. The output of compressor 31 is applied to compensator 31A. Compensator 31A includes compensation circuits to ensure that the open loop gain meets the Nyquist stability criteria so that the system will not oscillate when the loop is closed. The system shown is duplicated once each for the left and right ears.

Power amplifier 32 amplifies the signal from compensator 31A and energizes headphone driver 17 to provide an acoustic signal in cavity 12 that is combined with an outside noise signal that enters cavity 12 from a region represented as acoustic input terminal 25 to produce a combined acoustic pressure signal in cavity 12 represented as a circle 36 applied to and transduced by internal microphone 11. Internal microphone preamplifier 35 amplifies the transduced signal and delivers it to signal combiner 30.

With switch S1 connected to terminal S1B, a transduced signal from outside microphone 41 coupled through outside microphone preamplifier 42, preferably having a bandpass restricted to the speech spectrum, is delivered to signal combiner 30.

The invention has a number of advantages when used with an active noise reducing headset, such as the Bose QuietComfort 2 headphones having an attached microphone. The wearer, such as an airplane passenger, may move switch S1 to receive the transduced speech signal of a flight attendant, for example, that is clearly heard with the benefit of the active noise reduction. Moving the switch to connect to terminal S1A allows the user to listen to an electrical audio signal on input 24, such as music or the sound track of a movie or, alternatively, with no signal applied, enjoy the tranquility of significantly reduced noise in a noisy atmosphere, such as that of an aircraft.

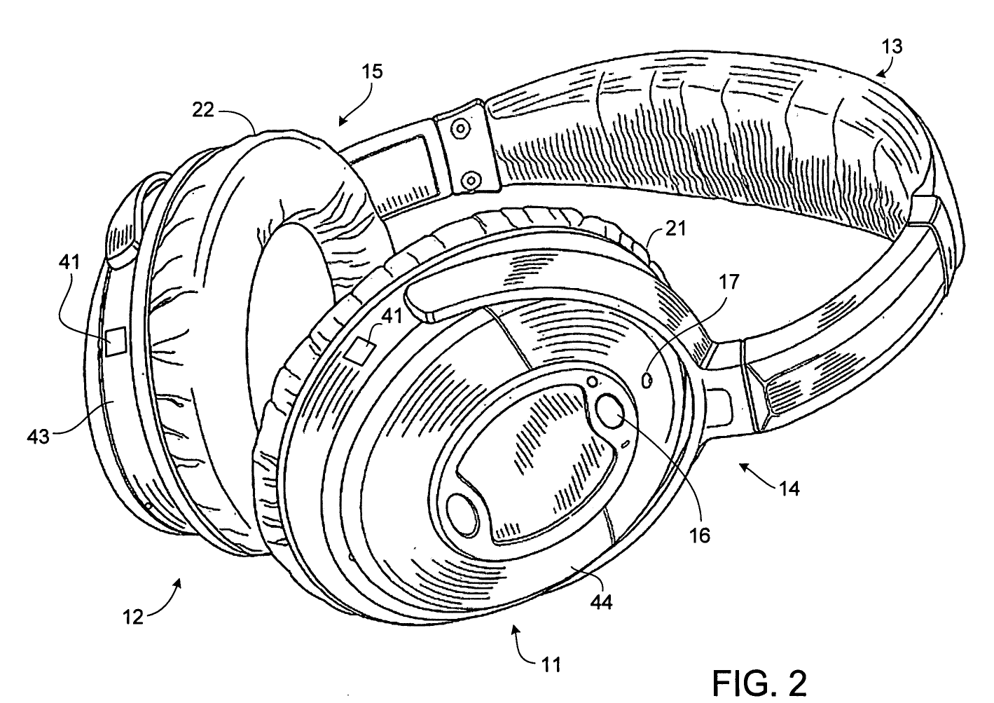

Referring to FIG. 2, there is shown a diagrammatic view of outside microphones, such as 41 or 41′, attached to headphone cups, such as 43 and 44.

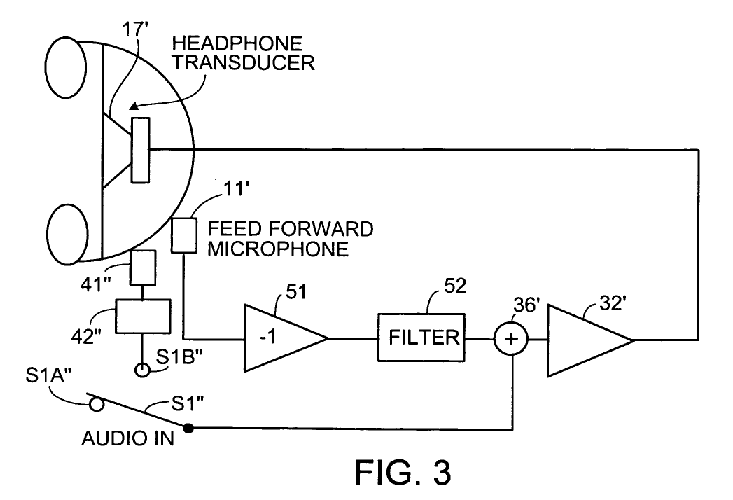

Referring to FIG. 3, there is shown a block-pictorial diagram of an embodiment of the invention using feed forward with an external feed forward microphone 11′ that is coupled by an inverting amplifier 51 and filter 52 that typically selectively transmits spectral components within a predetermined band of audio frequencies to effect noise reduction in that band. The output of filter 52 is coupled to one input of combiner 36′. The other input of combiner 36′ is coupled to the arm of switch S1 “that selectively receives either the audio input signal on terminal S1A” or the amplified sound signal transduced by outside microphone 41″ on terminal S1B″. The output of combiner 36′ is coupled to power amplifier 32′ that drives headphone driver 17′. Feed forward microphone 11′ is always coupled to an input of combiner 36′ to effect active noise reduction.

There has been described novel apparatus and techniques for improving communication in a noisy environment. It is evident that those skilled in the art may now make numerous uses and modifications of and departures from the specific apparatus and techniques herein disclosed without departing from the inventive concepts. Consequently, the invention is to be construed as embracing each and every novel feature and novel combination of features present in or possessed by apparatus and techniques herein disclosed and limited only by the spirit and scope of the appended claims.

What is claimed is:

1. Communication apparatus comprising,
a noise reducing headphone,
an input terminal for receiving an electrical input signal,
an outside microphone attached to said headphone constructed and arranged to electroacoustically transduce speech into a corresponding electrical speech signal,
a switch coupled to said headphone having a first terminal coupled to said input terminal and a second terminal coupled to said outside microphone constructed and arranged to selectively couple said headphone to only one of said first terminal and said second terminal,
and a noise reducing microphone for electroacoustically transducing an undesired acoustic signal into a corresponding undesired acoustic electrical signal,
a combiner constructed and arranged to combine the desired signal on that one of the first and second terminals which said switch has then selected with said undesired acoustic electrical signal to provide a combined signal,
and a power amplifier coupling said combined signal to said headphone to reduce the audibility of said undesired acoustic signal to a user of said headphone.

2. Communication apparatus in accordance with claim 1 wherein said noise-reducing microphone is inside said headphone and comprises a negative feedback circuit constructed and arranged to reduce the audibility of said undesired acoustic signal to a user of said headphone.

3. Communication apparatus in accordance with claim 1 wherein said noise-reducing microphone is outside said headphone and comprises a feed forward circuit constructed and arranged to reduce the audibility of said undesired acoustic signal to a user of said headphone.

4. Communication apparatus in accordance with claim 1 and further comprising a second headphone, wherein said switch is coupled to both said headphones to selectively couple said headphones to only one of said first terminal and said second terminal.

5. Communication apparatus in accordance with claim 4 and further comprising a second outside microphone attached to said second headphone constructed and arranged to electroaccoustically transduce speech into a corresponding electrical speech signal.
